(19) United States
(12) Patent Application Publication
(10) Pub. No.: US 2021/0244744 A1
(43) Pub. Date: Aug. 12, 2021

(54) TREATING LIVER DISORDERS

(71) Applicant: Terns Pharmaceuticals, Inc., Foster City, CA (US)

(72) Inventors: Randall HALCOMB, Foster City, CA (US); Weidong ZHONG, Foster City, CA (US); Martijn FENAUX, Foster City, CA (US)

(21) Appl. No.: 17/271,554

(22) PCT Filed: Aug. 30, 2018
(86) PCT No.: PCT/CN2018/103349

§ 371 Date: Feb. 25, 2021

(51) Int. Cl. A61K 31/55; A61K 31/42; A61K 31/445; A61P 1/16; A61K 47/38

(52) U.S. Cl. CPC A61K 31/55; A61K 31/42; A61K 47/38; A61P 1/16; A61K 31/445

(57) ABSTRACT

Provided herein are methods and compositions for treating liver disorders, including without limitation non-alcoholic steatohepatitis, and symptoms and manifestations thereof, in a patient. Accordingly, utilized herein are compounds of formulas (I), (II), etc., as disclosed herein.

### FIELD OF THE INVENTION

This invention relates to methods and compositions for treating liver disorder in a patient.

### STATE OF THE ART

There is a need to provide alternative therapies for liver disorders such as non-alcoholic fatty liver disease (NAFLD), or non-alcoholic steatohepatitis (NASH).

### SUMMARY

Provided herein are methods and compositions for treating a patient in need of treatment for a liver disorder. The method comprises administering to the patient a therapeutically effective amount of a compound of formulae I, II, or another compound utilized herein. In certain embodiments, liver disorders include, without limitation, liver inflammation, fibrosis, and steatohepatitis. In certain embodiments, the liver disorder is selected from: liver fibrosis, alcohol induced fibrosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc.

As used herein, Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis.

In one embodiment provided herein is a pharmaceutically acceptable composition comprising a compound of formula (I) or (II), or a tautomer thereof, or an isotopomer of each thereof, or an enantiomer or diastereomer of the foregoing, or a pharmaceutically acceptable salt of each of the above, and at least one pharmaceutically acceptable excipient, carrier, or diluent for treating a liver disorder; impeding or slowing the progression of non-alcoholic fatty liver disease (NAFLD) to non-alcoholic steatohepatitis (NASH); or for impeding or slowing the progression of NASH, in a patient in need thereof, wherein the liver disorder is selected from liver inflammation, liver fibrosis, alcohol induced fibrosis, steatosis, alcoholic steatosis, NAFLD, and NASH.

In one embodiment, provided herein is a unit dose form of the pharmaceutically acceptable formulations provided herein. In some embodiments, the unit dose form comprises a therapeutically effective amount of a compound of formula (I) or (II). In one embodiment, the unit dose form is for treating a liver disorder; of impeding or slowing the progression of non-alcoholic fatty liver disease (NAFLD) to non-alcoholic steatohepatitis (NASH); or of impeding or slowing the progression of NASH, in a patient in need thereof, wherein the liver disorder is selected from liver inflammation, liver fibrosis, alcohol induced fibrosis, steatosis, alcoholic steatosis, NAFLD, and NASH.

In one embodiment, the compound of formula (I) or (II) is the compound is: 4-{4-[5-cyclopropyl-3-(2,6-dichloro-phenyl)-isoxazol-4-ylmethoxy]-azepan-1-yl}-benzoic acid:

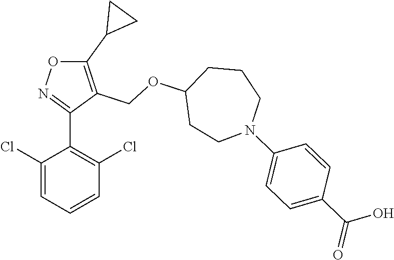 [SMILES: O=C(O)c1ccc(N2CCCC(OCc3c(-c4c(Cl)cccc4Cl)noc3C3CC3)CC2)cc1]

or a pharmaceutically acceptable salt or enantiomer thereof.

In one embodiment, the compound of formula (I) or (II) is trans-4-{4-[5-cyclopropyl-3-(2,6-dichloro-phenyl)-isoxazol-4-ylmethoxy]-cyclohexyl}-benzoic acid:

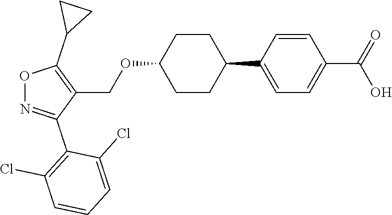 [SMILES: O=C(O)c1ccc([C@H]2CC[C@H](OCc3c(-c4c(Cl)cccc4Cl)noc3C3CC3)CC2)cc1]

or a pharmaceutically acceptable salt or enantiomer thereof.

In one embodiment, the compound of formula (I) or (II) is 6-{4-[5-cyclopropyl-3-(2,6-dichloro-phenyl)-isoxazol-4-ylmethoxy]-piperidin-1-yl}-1-methyl-1H-indole-3-carboxylic acid:

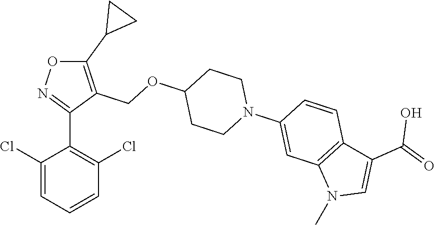 [SMILES: Cn1cc(C(=O)O)c2ccc(N3CCC(OCc4c(-c5c(Cl)cccc5Cl)noc4C4CC4)CC3)cc21]

or a pharmaceutically acceptable salt or enantiomer thereof.

In one embodiment, the therapeutically effective amount is 5 mg/day/patient-600 mg/day/patient. In another embodiment, the therapeutically effective amount is 75 mg/day/patient-600 mg/day/patient. In another embodiment, the therapeutically effective amount is about 25 mg/day/patient. In another embodiment, the therapeutically effective amount is about 75 mg/day/patient. In another embodiment, the therapeutically effective amount is about 200 mg/day/patient. In another embodiment, the therapeutically effective amount is about 400 mg/day/patient. In another embodiment, the therapeutically effective amount is about 600 mg/day/patient. In one embodiment, the compound of formula (I) or (II) is administered as a monotherapy, i.e., administered in absence of another agent, which: is useful in treating or substantially treating a liver disorder, impedes or slows the progression of non-alcoholic fatty liver disease (NAFLD) to non-alcoholic steatohepatitis (NASH); or impedes or slows the progression of NASH, in a patient in need thereof.

In one embodiment, the therapeutically effective amount is administered once daily. In one embodiment, the therapeutically effective amount of is administered twice daily. In one embodiment, the therapeutically effective amount is 75 mg-200 mg twice daily per patient. In one embodiment, the compound is administered as a pharmaceutically acceptable composition comprising at least one pharmaceutically acceptable excipient, carrier, or diluent.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than employing the corresponding drug. For illustration and without limitation, prodrugs include, carboxy esters, linear and cyclic phosphate esters and phosphoramide and phosphoramidates, carbamates, preferably phenolic carbamates (i.e., carbamates where the hydroxy group is part of an aryl or heteroaryl moiety, where the aryl and heteroaryl may be optionally substituted), and the likes.

“Salt” refers to an ionic compound formed between an acid and abase. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

An “isotopomer” of a compound is a compound in which one or more atoms of the compound have been replaced with isotopes of those same atoms. For example, where H has been replaced by D or T, or ¹²C has been replaced by ¹¹C or ¹⁴N has been replaced by ¹⁵N. For example, and without limitation, replacement of with D can in some instances lead to reduced rates of metabolism and therefore longer half-lives. Replacement of H with T can provide radioligands potentially useful in binding studies. Replacement of ¹²C with the short-lived isotope ¹¹C can provide ligands useful in Positron Emission Tomography (PET) scanning. Replacement of ¹⁴N with ¹⁵N provides compounds that can be detected/monitored by ¹⁵N NMR spectroscopy. For example, and without limitation, an isotopomer of a compound containing —CH₂CH₃ is that compound but containing —CD₂CD₃ instead of the —CH₂CH₃.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkylene” refers to a divalent saturated aliphatic hydrocarbyl group having from 1 to 12 carbon atoms, preferably from 1 to 10 carbon atoms, and more preferably from 1 to 6 carbon atoms. This term includes, by way of example, linear and branched hydrocarbyl groups such as methylene (—CH₂—), ethylene (—CH₂CH₂— or —CH(Me)-), propylene (—CH₂CH₂CH₂— or —CH(Me)CH₂—, or —CH(Et)-) and the likes.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C═CH), and propargyl (—CH₂C≡CH). Cₓ alkynyl refers to an alkynyl group having x number of carbon atoms.

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein and with the proviso that any hydroxyl or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein. Preferred substituted alkyl groups in —O-(substituted alkyl) include halogenated alkyl groups and particularly halogenated methyl groups such as trifluoromethyl, difluromethyl, fluoromethyl and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein. Acyl includes the “acetyl” group CHC(O)—.

“Acylamino” refers to the groups —NR³⁰C(O)alkyl, —NR³⁰C(O)substituted alkyl, —NR³⁰C(O)cycloalkyl, —NR³⁰C(O)substituted cycloalkyl, —N R³⁰C(O)alkenyl, —NR³⁰C(O)substituted alkenyl, alkoxy, substituted alkoxy-NR³⁰C(O)alkynyl, —NR³⁰C(O)substituted alkynyl, —NR³⁰C(O)aryl, —NR³⁰C(O)substituted aryl, —NR³⁰C(O)heteroaryl, —NR³⁰C(O)substituted heteroaryl, —NR³⁰C(O)heterocyclic, and —NR³⁰C(O)substituted heterocyclic wherein R³⁰ is hydrogen, alkyl, substituted alkyl, aryl, substituted aryl, heteroaryl, substituted heteroaryl, cycloalkyl, or substituted cycloalkyl; and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³¹R³² where R³¹ and R³² are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, substituted heterocyclic, arylamino, substituted arylamino, heteroarylamino, substituted heteroarylamino, cycloalkylamino, substituted cycloalkylamino, heterocycloalkylamino, substituted heterocyclylamino, sulfonylamino, and substituted sulfonyl and wherein R³¹ and R³² are optionally joined, together with the nitrogen bound thereto to form a heterocyclic or substituted heterocyclic group, provided that R³¹ and R³² are both not hydrogen, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic, and substituted heterocyclic are as defined herein. When R³¹ is hydrogen and R³² is alkyl, the substituted amino group is sometimes referred to herein as alkylamino. When R³¹ and R³² are alkyl, the substituted amino group is sometimes referred to herein as dialkylamino. When referring to a monosubstituted amino, it is meant that either R³¹ or R³² is hydrogen but not both. When referring to a disubstituted amino, it is meant that neither R³¹ nor R³² are hydrogen.

“Aminocarbonyl” refers to the group —C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonyl” refers to the group —C(S)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR³⁰C(O)NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the group —NR³⁰C(S)NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the group —O—C(O)NR³³R³³ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyl” refers to the group —SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyloxy” refers to the group —O—SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonylamino” refers to the group —NR³⁰—SO₂NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Amidino” refers to the group —C(═NR³⁵)NR³³R³⁴ where R³³, R³⁴, and R³⁵ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Arylamino” refers to the group —NR³⁷(aryl), where aryl is as defined herein and R³⁷ is hydrogen, alkyl, or substituted alkyl.

“Substituted arylamino” refers to the group —NR³⁷(substituted aryl), where R³⁷ is hydrogen, alkyl, or substituted alkyl where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR³⁰—C(O)O-alkyl, —NR³⁰—C(O)O-substituted alkyl, —NR³⁰—C(O)O-alkenyl, —NR³⁰—C(O)O-substituted alkenyl, —NR³⁰—C(O)O-alkynyl, —NR³⁰—C(O)O-substituted alkynyl, —NR³⁰—C(O)O-aryl, —NR³—C(O)O-substituted aryl, —NR³⁰—C(O)O-cycloalkyl, —NR³⁰—C(O)O-substituted cycloalkyl, —NR³⁰—C(O)O-heteroaryl, —NR³⁰—C(O)O-substituted heteroaryl, —NR³⁰—C(O)O-heterocyclic, and —NR³⁴—C(O)O-substituted heterocyclic wherein R³⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylamino” refers to the group —NR³⁷(cycloalkyl) where R³⁷ is hydrogen, alkyl, or substituted alkyl.

“Substituted cycloalkylamino” refers to the group —NR³⁷(substituted cycloalkyl) where R³⁷ is hydrogen, alkyl, or substituted alkyl and substituted cycloalkyl is as defined herein.

“Cycloalkylthio” refers to —S-cycloalkyl.

“Substituted cycloalkylthio” refers to —S-(substituted cycloalkyl).

“Guanidino” refers to the group —NHC(═NH)NH₂.

“Substituted guanidino” refers to —NR³⁶C(═NR³⁶)N(R³⁶)₂ where each R³⁶ is independently selected from the group consisting of hydrogen, alkyl, substituted alkyl, aryl, substituted aryl, heteroaryl, substituted heteroaryl, heterocyclic, and substituted heterocyclic and two R³⁶ groups attached to a common guanidino nitrogen atom are optionally joined together with the nitrogen bound thereto to form a heterocyclic or substituted heterocyclic group, provided that at least one R³⁶ is not hydrogen, and wherein said substituents are as defined herein.

“Halo” or “halogen” refers to fluoro, chloro, bromo and iodo and preferably is fluoro or chloro.

“Hydroxy” or “hydroxyl” refers to the group —OH.

“Heteroalkylene” refers to an alkylene group wherein one or more carbons is replaced with —O—, —S—, SO₂, a P containing moiety as provided herein, —NR^{Q}—,

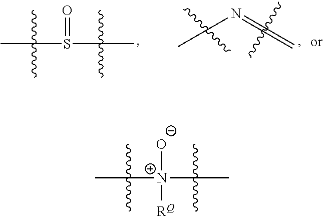

moieties where R^{Q} is H or C—C₆ alkyl. “Substituted heteroalkylene” refers to heteroalkynylene groups having from 1 to 3 substituents, and preferably 1 to 2 substituents, selected from the substituents disclosed for substituted alkylene.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heteroarylamino” refers to the group —NR³⁷(heteroaryl) where R³⁷ is hydrogen, alkyl, or substituted alkyl.

“Substituted heteroarylamino” refers to the group —NR³⁷ (substituted heteroaryl), where R³⁷ is hydrogen, alkyl, or substituted alkyl and substituted heteroaryl is defined as herein.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Heterocyclylene” refers to a divalent saturated or partially saturated, but not aromatic, group having from 1 to 10 ring carbon atoms and from 1 to 4 ring heteroatoms selected from the group consisting of nitrogen, sulfur, or oxygen. “Substituted heterocyclylene” refers to heterocyclylene groups that are substituted with from 1 to 5 or preferably 1 to 3 of the same substituents as defined for substituted cycloalkyl

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

“Heterocyclylamino” refers to the group —NR³⁷(heterocyclyl) where R³⁷ is hydrogen, alkyl, or substituted alkyl.

“Substituted heterocyclylamino” refers to the group —NR³⁷ (substituted heterocyclyl), where R³⁷ is hydrogen, alkyl, or substituted alkyl and substituted heterocyclyl is defined as herein.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Nitro” refers to the group —NO₂.

“Oxo” refers to the atom (═O) or (O).

“Spiro ring systems” refers to bicyclic ring systems that have a single ring carbon atom common to both rings.

“Sulfinyl” refers to the divalent group —S(O)— or —S(═O)—.

“Sulfonyl” refers to the divalent group —S(O)₂— or —S(═O)₂—.

“Substituted sulfonyl” refers to the group —SO₂-alkyl, —SO₂-substituted alkyl, —SO₂—OH, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂. Preferred substituted alkyl groups on the substituted alkyl-SO₂— include halogenated alkyl groups and particularly halogenated methyl groups such as trifluoromethyl, difluromethyl, fluoromethyl and the like.

“Substituted sulfinyl” refers to the group —SO-alkyl, —SO-substituted alkyl, —SO-alkenyl, —SO-substituted alkenyl, —SO-cycloalkyl, —SO-substituted cycloalkyl, —SO-aryl, —SO-substituted aryl, —SO-heteroaryl, —SO-substituted heteroaryl, —SO-heterocyclic, —SO-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfinyl includes groups such as methyl-SO—, phenyl-SO—, and 4-methylphenyl-SO—. Preferred substituted alkyl groups on the substituted alkyl-SO— include halogenated alkyl groups and particularly halogenated methyl groups such as trifluoromethyl, difluromethyl, fluoromethyl and the like.

“Sulfonyloxy” or “substituted sulfonyloxy” refers to the group —OSO₂-alkyl, —OSO₂-substituted alkyl, —OSO₂—OH, —OSO₂-alkenyl, —OSO₂-substituted alkenyl, —OSO₂-cycloalkyl, —OSO₂-substituted cycloalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Sulfonylamino” refers to the group —NR³⁷(substituted sulfonyl) where R³⁷ is hydrogen, alkyl, or substituted alkyl and substituted sulfonyl is as defined here.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Mercapto” or “thiol” refers to the group —SH.

“Formyl” refers to the group —C(O)H.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein. Preferred substituted alkyl groups on —S-(substituted alkyl) include halogenated alkyl groups and particularly halogenated methyl groups such as trifluoromethyl, difluromethyl, fluoromethyl and the like.

“Vinyl” refers to unsaturated hydrocarbon radical —CH═CH₂, derived from ethylene.

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

The term “optionally substituted” refers to a substituted or unsubstituted group. The substituted group may be substituted with one or more substituents, such as e.g., 1, 2, 3, 4 or 5 substituents. Preferably, the substituents are selected from the functional groups provided herein. In certain more preferred embodiments, the substituents are selected from oxo, halo, —CN, NO₂, —CO₂R⁵⁰, —OR⁵⁰, —SR⁵⁰, —SOR⁵⁰, —SO₂R⁵⁰, —NR⁵¹R⁵², —CONR⁵¹R⁵², —SO₂NR⁵¹R⁵², C₁-C₆ alkyl, C₁-C₆ alkoxy, —CR⁵⁰═C(R⁵⁰)₂, —CCR⁵⁰, C₃-C₁₀ cycloalkyl, C₄-C₁₀ heterocyclyl, C₆-C₁₄ aryl and C₅-C₁₂ heteroaryl, wherein each R⁵⁰ independently is hydrogen or C₁-C₈ alkyl; C₃-C₁₂ cycloalkyl; C₄-C₁₀ heterocyclyl; C₆-C₁₄ aryl; or C₂-C₁₂ heteroaryl; wherein each alkyl, cycloalkyl, heterocyclyl, aryl, or heteroaryl is optionally substituted with 1-3 halo, 1-3 C₁-C₆ alkyl, 1-3 C₁-C₆ haloalkyl or 1-3 C₁-C₆ alkoxy groups. More preferably, the substituents are selected from the group consisting of chloro, fluoro, —OCH₃, methyl, ethyl, iso-propyl, cyclopropyl, —OCF₃, —CF₃ and —OCHF₂.

R⁵¹ and R⁵² independently are hydrogen; C₁-C₈ alkyl, optionally substituted with —CO₂H or an ester thereof, C₁-C₆ alkoxy, oxo, —CR⁵³═C(R⁵³)₂, —CCR⁵³, C₃-C₁₀ cycloalkyl, C₃-C₁₀ heterocyclyl, C₆-C₁₄ aryl, or C₂-C₁₂ heteroaryl, wherein each R⁵ independently is hydrogen or C₁-C₈ alkyl; C₃-C₁₂ cycloalkyl; C₄-C₁₀ heterocyclyl; C₆-C₁₄ aryl; or C₂-C₁₂ heteroaryl; wherein each cycloalkyl, heterocyclyl, aryl, or heteroaryl is optionally substituted with 1-3 alkyl groups or 1-3 halo groups, or R⁵ and R⁵² together with the nitrogen atom they are attached to form a 5-7 membered heterocycle.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

It is understood that the above definitions are not intended to include impermissible substitution patterns (e.g., methyl substituted with 4 fluoro groups). Such impermissible substitution patterns are well known to the skilled artisan.

### Descriptive Embodiments

In one embodiment, the compound utilized herein is of formula (I):

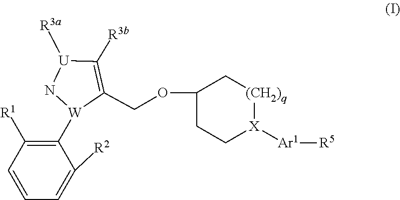

wherein: q is 1 or 2;

### U is O, N or C;

W is C or N; provided that when U is O or N, R^{3a} is absent; and provided that when U is N or C, the UN bond is a double bond; and provided that when W is C, the WN bond is a double bond;

### X is CH or N;

R¹ is halo or C₁-C₃ alkoxy optionally substituted with 1-5 halo, preferably fluoro atoms;
R² is hydrogen, halo or C₁-C₃ alkoxy optionally substituted with 1-5 halo, preferably fluoro atoms;
R^{3a} is hydrogen, or is absent;
R^{3b} is C₁-C₃ alkyl optionally substituted with 1-5 halo, preferably fluoro atoms; or is C₃-C₄ cycloalkyl optionally substituted with 1-3 methyl or ethyl groups; or is a 4 membered heterocyclyl optionally substituted with 1-3 methyl or ethyl groups;
Ar¹ is selected from optionally substituted 6-10 member aryl and optionally substituted 5-10 membered heteroaryl; and
R⁵ is COOH or a carboxylic acid isostere;
or a tautomer thereof, or an isotopomer of each thereof, or an enantiomer or diastereomer of the foregoing, or a pharmaceutically acceptable salt of each of the above.

In another embodiment, the compound utilized herein is of formula (I), wherein:

q is 1 or 2, provided that when X is CH, q is 1;
U is O, N or C; provided that when U is O or N, R³, is absent; and provided that when U is N or C, the UN bond is a double bond; and provided that when W is C, the WN bond is a double bond;

### W is C or N;

### X is CH or N;

R¹ is chloro, fluoro, or trifluoromethoxy;
R² is hydrogen chloro, fluoro, or trifluoromethoxy;
R^{3a} is hydrogen, or is absent;
R^{3b} is trifluoromethyl, cyclopropyl or isopropyl;
Ar¹ is selected from optionally substituted indolyl, optionally substituted benzothienyl, optionally substituted naphthyl, optionally substituted phenyl, optionally substituted benzoisothiazolyl, optionally substituted indazolyl, and optionally substituted pyridinyl; preferably, indolyl, benzothienyl, naphthyl, phenyl, benzoisothiazolyl, indazolyl, and pyridinyl, each optionally substituted with a group selected from methyl, ethyl, and phenyl; more preferably 6-indolyl, 6-benzothienyl, 4-naphthyl, 4-phenyl, and 2-pyridinyl, each optionally substituted with one or two groups independently selected from methyl, ethyl, and phenyl; yet more preferably 4-phenyl, 6-indolyl or 6-benzothienyl, each optionally substituted with methyl or phenyl; and

### R⁵ is COOH;

or tautomer thereof, or an isotopomer of each thereof, or an enantiomer or diastereomer of the foregoing, or a pharmaceutically acceptable salt of each of the above.

In one embodiment, the compound utilized herein is of formula (II):

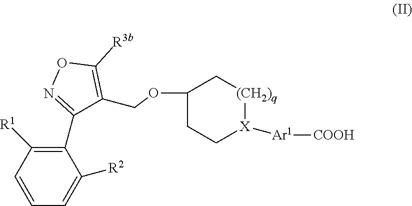

wherein the variables are defined as herein.

In one embodiment, the compound utilized herein is of formula (I), wherein:

q is 1 or 2;
R¹ is chloro, fluoro, or trifluoromethoxy;
R² is hydrogen chloro, fluoro, or trifluoromethoxy;
R^{3b} is trifluoromethyl, cyclopropyl or isopropyl; X is CH or N, provided that when X is CH, q is 1; and
Ar¹ is selected from indolyl, benzothienyl, naphthyl, phenyl, benzoisothiazolyl, indazolyl, and pyridinyl, each optionally substituted with methyl or phenyl.

In certain embodiments, U is O and W is C, and together form an isoxazole ring:

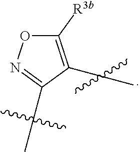

In certain embodiments, U and W are both N, and together form a triazole ring:

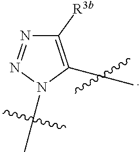

In certain embodiments, U is C and W is N, and together form a pyrazole ring:

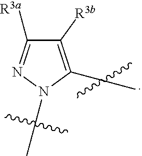

In one embodiment, wherein R^{3b} is cyclopropyl or isopropyl. In one embodiment, R^{3b} is cyclopropyl.

In one embodiment, R¹ is chloro or trifluoromethoxy and R² is hydrogen or chloro. In one embodiment, R¹ and R² are both chloro or R¹ is trifluoromethoxy and R² is hydrogen.

In one embodiment, R¹ is chloro. In one embodiment, R¹ is trifluoromethoxy

In one embodiment, R² is chloro. In one embodiment, R² is H.

In one embodiment, R^{3b} is cyclopropyl. In one embodiment, R^{3b} is isopropyl.

In certain embodiments, R¹ is chloro or trifluoromethoxy; R² is hydrogen or chloro; R^{3a} is hydrogen or absent; R^{3b} is cyclopropyl or isopropyl and Ar¹ is 4-phenyl, 2-pyridinyl, 6-indolyl, or 6-benzothienyl each optionally substituted with a group selected from methyl, trifluoromethyl or phenyl.

In certain embodiments, Ar¹ is selected from optionally substituted indolyl, optionally substituted benzothienyl, optionally substituted naphthyl, optionally substituted phenyl, optionally substituted benzoisothiazolyl, optionally substituted indazolyl, and optionally substituted pyridinyl. In certain embodiments, Ar¹ is selected from indolyl, benzothienyl, naphthyl, phenyl, benzoisothiazolyl, indazolyl, and pyridinyl, each optionally substituted with a group selected from methyl, ethyl, and phenyl. In certain embodiments, Ar¹ is optionally substituted 4-phenyl. In one embodiment, Ar¹ is optionally substituted 2-pyridinyl. In one embodiment, Ar¹ is optionally substituted 6-benzothienyl. In certain embodiments, preferably Ar¹ is optionally substituted with a group selected from methyl, ethyl and phenyl. A more preferred optional substituent is methyl. In certain embodiments, Ar¹ is selected from 6-indolyl, 6-benzothienyl, 4-naphthyl, 4-phenyl, and 2-pyridinyl, each optionally substituted with one or two groups independently selected from methyl, ethyl, and phenyl. In certain embodiments, Ar is selected from 4-phenyl, 6-indolyl or 6-benzothienyl, each optionally substituted with methyl or phenyl. In one embodiment, Ar¹ is 4-phenyl. In some embodiments, the 4-phenyl is substituted as disclosed herein. In one embodiment, Ar¹ is 6-indolyl. In some embodiments, the 6-indolyl is substituted as disclosed herein. In one embodiment, Ar is 6-benzothienyl. In some embodiments, the 6-benzothienyl is substituted as disclosed herein. As will be apparent to the skilled artisan, the Ar¹ moiety is a divalent moiety, and the aryl and heteroaryl groups representing the Ar¹ moities are also divalent.

In certain embodiments, q is 1; R¹ is chloro or trifluoromethoxy; R² is hydrogen or chloro; R^{3b} is cyclopropyl and Ar¹ group is 4-phenyl, 2-pyridinyl, or 6-indolyl, each optionally substituted with methyl. Also preferred is a compound wherein q is 2; R¹ is chloro or trifluoromethoxy; R² is hydrogen or chloro; R^{3b} is cyclopropyl; X is N and Ar¹ group is A-phenyl, 2-pyridinyl, or 6-indolyl, each optionally substituted with methyl.

In certain embodiments, U is oxygen, and W is carbon forming an isoxazole ring; R¹ is chloro or trifluoromethoxy; R² is hydrogen or chloro; R^{3a} is absent and R^{3b} is cyclopropyl and Ar¹ group is 4-phenyl, 2-pyridinyl, 6-indolyl or 6-benzothienyl each optionally substituted with methyl.

In certain embodiments, U and W are both nitrogen forming a triazole ring; R¹ is chloro or trifluoromethoxy; R² is hydrogen or chloro; R^{3a} is absent and R^{3b} is cyclopropyl or isopropyl and Ar¹ group is 4-phenyl, 6-indolyl or 6-benzothienyl, each optionally substituted with methyl or phenyl.

In certain embodiments, U is carbon, W is nitrogen forming a pyrazole ring; R¹ is chloro or trifluoromethoxy; R² is hydrogen or chloro; R^{3a} is hydrogen and R^{3b} is cyclopropyl, or isopropyl and Ar¹ group is 4-phenyl, 6-indolyl or 6-benzothienyl, each optionally substituted with methyl or phenyl.

In certain embodiments, q is 1; U is oxygen, and W is carbon forming an isoxazole ring; R¹ is chloro or trifluoromethoxy; R² is hydrogen or chloro; R^{3a} is absent and R^{3b} is cyclopropyl; X is CH and Ar¹ group is 4-phenyl, 2-pyridinyl, 6-indolyl or 6-benzothienyl each optionally substituted with methyl.

In certain embodiments, q is 1; U and W are both nitrogen forming a triazole ring; R¹ is chloro or trifluoromethoxy; R² is hydrogen or chloro; R^{3a} is absent and R^{3b} is cyclopropyl or isopropyl; X is CH and Ar¹ group is A-phenyl, 6-indolyl or 6-benzothienyl, each optionally substituted with methyl or phenyl.

In certain embodiments, q is 1; U is carbon, W is nitrogen forming a pyrazole ring; R¹ is chloro or trifluoromethoxy; R² is hydrogen or chloro; R^{3a} is hydrogen and R^{3b} is cyclopropyl, or isopropyl; X is CH and Ar¹ group is A-phenyl, 6-indolyl or 6-benzothienyl, each optionally substituted with methyl or phenyl.

In certain embodiments, U is oxygen, and W is carbon forming an isoxazole ring; R¹ is chloro or trifluoromethoxy; R² is hydrogen or chloro; R^{3a} is absent and R^{3b} is cyclopropyl; X is N and Ar₁ group is 4-phenyl, 2-pyridinyl, 6-indolyl or 6-benzothienyl each optionally substituted with methyl.

In certain embodiments, U and W are both nitrogen forming a triazole ring; R¹ is chloro or trifluoromethoxy; R² is hydrogen or chloro; R^{3a} is hydrogen and R^{3b} is cyclopropyl or isopropyl; X is N and Ar group is 4-phenyl, 6-indolyl or 6-benzothienyl, each optionally substituted with methyl or phenyl.

In certain embodiments, U is carbon, W is nitrogen forming a pyrazole ring; R¹ is chloro or trifluoromethoxy; R² is hydrogen or chloro; R^{3a} is hydrogen and R^{3b} is cyclopropyl, or isopropyl; X is N and Ar¹ group is 4-phenyl, 6-indolyl or 6-benzothienyl, each optionally substituted with methyl or phenyl.

In one embodiment, Ar¹ is 6-benzoisothiazolyl, 5-benzothienyl, 6-benzothienyl, 6-indazolyl, 5-indolyl or 6-indolyl, 4-phenyl and 2-pyridinyl, each optionally substituted with methyl or phenyl. Preferably Ar¹ is 6-benzoisothiazolyl, 5-benzothienyl, 6-benzothienyl, 6-indazolyl, 5-indolyl, 6-indolyl, or 4-phenyl, each optionally substituted with methyl. Most preferably Ar¹ group is 5-benzothienyl, 6-benzothienyl, 5-indolyl, 6-indolyl or 4-phenyl, each optionally substituted with methyl.

In one embodiment, q is 1 and X is N.

In one embodiment, q is 1 and X is CH.

In one embodiment, q is 2 and X is N.

In some embodiments, examples of carboxylic acid isosteres include, without limitation, 1-H tetrazole, boronic acid, hydroxamic acid, phosphonic acid, and squaric acid.

In one embodiment, the compound utilized herein is selected from:
5-{4-[5-Cyclopropyl-3-(2,6-dichloro-phenyl)-isoxazol-4-ylmethoxy]-piperidin-1-yl}-biphenyl-2-carboxylic acid,
5-{4-[5-Cyclopropyl-3-(2-trifluoromethoxy-phenyl)-isoxazol-4-ylmethoxy]-piperidin-1-yl}-biphenyl-2-carboxylic acid,
5-{4-[3-(2,6-Dichloro-phenyl)-5-isopropyl-isoxazol-4-ylmethoxy]-piperidin-1-yl}-biphenyl-2-carboxylic acid,
4-{4-[3-(2,6-Dichloro-phenyl)-5-isopropyl-isoxazol-4-ylmethoxy]-piperidin-1-yl}-naphthalene-1-carboxylic acid,
4-{4-[5-Cyclopropyl-3-(2,6-dichloro-phenyl)-isoxazol-4-ylmethoxy]-piperidin-1-yl}-3-methyl-benzoic acid,
4-{4-[5-Isopropyl-3-(2-trifluoromethoxy-phenyl)-isoxazol-4-ylmethoxy]-piperidin-1-yl}-benzoic acid,
4-{4-[5-Cyclopropyl-3-(2,6-dichloro-phenyl)-isoxazol-4-ylmethoxy]-piperidin-1-yl}-benzoic acid,
4-{4-[5-Cyclopropyl-3-(2-trifluoromethoxy-phenyl)-isoxazol-4-ylmethoxy]-piperidin-1-yl}-2-methyl-benzoic acid,
4-{4-[5-Cyclopropyl-3-(2,6-dichloro-phenyl)-isoxazol-4-ylmethoxy]-piperidin-1-yl}-2-methyl-benzoic acid,
4-{4-[3-(2,6-Dichloro-phenyl)-5-isopropyl-isoxazol-4-ylmethoxy]-piperidin-1-yl}-2-methyl-benzoic acid,
4-{4-[3-(2,6-Dichloro-phenyl)-5-isopropyl-isoxazol-4-ylmethoxy]-piperidin-1-yl}-benzoic acid,
6-{4-[3-(2,6-Dichloro-phenyl)-5-isopropyl-isoxazol-4-ylmethoxy]-piperidin-1-yl}-1-methyl-1H-indole-3-carboxylic acid,
6-{4-[3-(2,6-Dichloro-phenyl)-5-isopropyl-isoxazol-4-ylmethoxy]-piperidin-1-yl}-benzo[b]thiophene-3-carboxylic acid,
6-{4-[5-Cyclopropyl-3-(2-trifluoromethoxy-phenyl)-isoxazol-4-ylmethoxy]-piperidin-1-yl}-1-methyl-1H-indole-3-carboxylic acid,
6-{4-[5-Cyclopropyl-3-(2,6-dichloro-phenyl)-isoxazol-4-ylmethoxy]-piperidin-1-yl}-1-methyl-1H-indole-3-carboxylic acid,
6-{4-[5-Cyclopropyl-3-(2,6-dichloro-phenyl)-isoxazol-4-ylmethoxy]-piperidin-1-yl}-benzo[b]thiophene-3-carboxylic acid,
4-[5-Cyclopropyl-3-(2,6-dichloro-phenyl)-isoxazol-4-ylmethoxy]-3,4,5,6-tetrahydro-2H-[1,2′]bipyridinyl-5′-carboxylic acid,
4-{4-[3-(2,6-Dichloro-phenyl)-5-isopropyl-isoxazol-4-ylmethoxy]-piperidin-1-yl}-3-methyl-benzoic acid,
4-{4-[5-Cyclopropyl-3-(2,6-dichloro-phenyl)-isoxazol-4-ylmethoxy]-azepan-1-yl}-benzoic acid,
6-{4-[5-Cyclopropyl-3-(2,6-dichloro-phenyl)-isoxazol-4-ylmethoxy]-azepan-1-yl}-1-methyl-1H-indole-3-carboxylic acid,
6-{4-[5-Cyclopropyl-3-(2,6-dichloro-phenyl)-isoxazol-4-ylmethoxy]-azepan-1-yl}-benzo[b]thiophene-3-carboxylic acid,
Trans-4-{4-[5-Cyclopropyl-3-(2,6-dichloro-phenyl)-isoxazol-4-ylmethoxy]-cyclohexyl}-benzoic acid,
Trans-4-{4-[5-Cyclopropyl-3-(2-trifluoromethoxy-phenyl)-isoxazol-4-ylmethoxy]-cyclohexyl}-benzoic acid,
Trans-6-{4-[5-Cyclopropyl-3-(2-trifluoromethoxy-phenyl)-isoxazol-4-ylmethoxy]-cyclohexyl}-1-methyl-1H-indole-3-carboxylic acid, and
Cis-6-{4-[5-Cyclopropyl-3-(2-trifluoromethoxy-phenyl)-isoxazol-4-ylmethoxy]-cyclohexyl}-1-methyl-1H-indole-3-carboxylic acid;
   or a pharmaceutically acceptable salt or enantiomer thereof.

The compounds utilized herein may be prepared by a combination of a variety of stepwise procedures known in the art, such as, e.g., US 2010/0152166 (incorporated herein by reference).

### Pharmaceutical Compositions and Formulations

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described.

Compositions comprising a compound provided herein are also described. In one variation, the composition comprises a compound and a pharmaceutically acceptable carrier or excipient. In another variation, a composition of substantially pure compound is provided.

### EXAMPLES

### Example 1: Mouse Model of NASH and Fibrosis Induced by 3H Diet

Male C57BL/6N mice are fed with D09100301 diet (Research Diets, 40% fat, 2% cholesterol, 24% fructose, (the, high fat, high cholesterol and high fructose, the “3H diet”) for 150 days. Each mouse is then singly housed after 5 days for an acclimation period. Plasma alanine aminotransferase (ALT) and cytokeratin 18 (CK18) are measured. After one week of recovery, the mice are randomized into 5 groups based on their ALT values, CK18 values, and body weight. Animals of each group are administrated either vehicle (0.5% methylcellulose (MC)+0.25% Tween 80 in distilled water) or a compound utilized herein (e.g., and without limitation at a dose such as 0.01-20 mg/kg) once daily in a volume of 5 ml/kg for 11 weeks.

Blood is collected from mice treated with a compound utilized herein for 76 days 2 hours after the last dose. Compound levels in the plasma are analyzed by mass spectroscopy. ALT, which indicates hepatic lesions in animals, is measured.

At the completion of the study, the animals are sacrificed and their livers excised. Two sections of the left and right lobes are fixed in neutral buffered 10% formalin. Liver tissue slides are stained with hematoxylin and eosin (H&E), Sirius red, and Masson's Trichrome to prepare slides for pathological analysis. All specimens are examined microscopically and scored as a modified Brunt score NASH Activity Score. Scores are based on the grading scheme and end-points as described in Brunt E. M, et al., “Histopathology of nonalcoholic fatty liver disease,” World J. of Gastroenterol, 2010, 16(42), 5286-5296. Group means are then calculated for each individual end-point. The following endpoints are used to characterize the fast food model of NASH in mice as modified from NASH endpoints (See Brunt, E. M. “Histopathology of nonalcoholic fatty liver disease,” Clin Liver Dis., 2009, 13, 533-544 and Brunt, E. M, et al., “Nonalcoholic steatohepatitis: A proposal for grading and staging the histological lesions”, Am. J. Gastroenterology, 1999, 94(9), 2467-2474.

Histopathological analysis of the livers from the mice treated with a compound utilized herein is performed. Hepatic inflammation, macrovesicular vaculation, and perisinusoidal fibrosis in the mice are measured and observed.

### Example 2: Treatments of Patients with NASH

Patients diagnosed with NASH and liver fibrosis stages F1, F2, F3, or F4, preferably F2 to F3 based on biopsy, or by magnetic resonance elastography (MRE) and MRI proton density fat fraction (MRI-PDFF) are divided into two groups and treated with either a compound of formula (I) or (II), (e.g., patient no., n=20) at about 75 mg-600 mg once daily or twice daily for 12 weeks, or treated with placebo. A decrease in liver fat content (measured by MRI-PDFF), improvement in liver biochemistry, and/or markers of fibrosis are measured.

1. A methods of treating a liver disorder; of impeding or slowing the progression of non-alcoholic fatty liver disease (NAFLD) to non-alcoholic steatohepatitis (NASH): or of impeding or slowing the progression of NASH, in a patient in need thereof, the method comprising administering to the patient a therapeutically effective amount of a compound of formula (I): 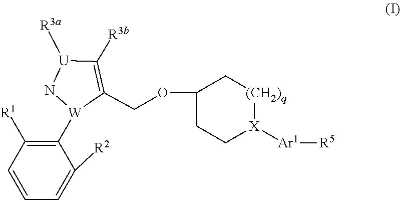
wherein: q is 1 or 2;
U is O, N or C;
W is C or N; provided that when U is O or N, R^{3a} is absent; and provided that when U is N or C, the UN bond is a double bond; and provided that when W is C, the WN bond is a double bond;
X is CH or N;
R¹ is halo or C₁-C₃ alkoxy optionally substituted with 1-5 halo, preferably fluoro atoms;
R² is hydrogen, halo or C₁-C₃ alkoxy optionally substituted with 1-5 halo, preferably fluoro atoms;
R^{3a} is hydrogen, or is absent;
R^{3b} is C₁-C₃ alkyl optionally substituted with 1-5 halo, preferably fluoro atoms; or is C₃-C₄ cycloalkyl optionally substituted with 1-3 methyl or ethyl groups; or is a 4 membered heterocyclyl optionally substituted with 1-3 methyl or ethyl groups;
Ar¹ is selected from optionally substituted 6-10 member aryl, optionally substituted 5-10 membered heteroaryl; and
R⁵ is COOH or a carboxylic acid isostere;
or a tautomer thereof, or an isotopomer of each thereof, or an enantiomer or diastereomer of the foregoing, or a pharmaceutically acceptable salt of each of the above;
wherein the liver disorder is selected from liver inflammation, liver fibrosis, alcohol induced fibrosis, alcoholic steatosis, NAFLD, and NASH.

2. The method of claim 1, wherein:
q is 1 or 2, provided that when X is CH, q is 1;
U is O, N or C; provided that when U is O or N, R^{3a} is absent; and provided that when U is N or C, the UN bond is a double bond; and provided that when W is C, the WN bond is a double bond;
W is C or N;
X is CH or N;
R¹ is chloro, fluoro, or trifluoromethoxy;
R² is hydrogen chloro, fluoro, or trifluoromethoxy;
R^{3a} is hydrogen, or absent;
R^{3b} is trifluoromethyl, cyclopropyl or isopropyl;
Ar¹ is selected from optionally substituted indolyl, optionally substituted benzothienyl, optionally substituted naphthyl, optionally substituted phenyl, optionally substituted benzoisothiazolyl, optionally substituted indazolyl, and optionally substituted pyridinyl; preferably, indolyl, benzothienyl, naphthyl, phenyl, benzoisothiazolyl, indazolyl, and pyridinyl, each optionally substituted with a group selected from methyl, ethyl, and phenyl; more preferably 6-indolyl, 6-benzothienyl, 4-naphthyl, 4-phenyl, and 2-pyridinyl, each optionally substituted with one or two groups independently selected from methyl, ethyl, and phenyl; yet more preferably 4-phenyl, 6-indolyl or 6-benzothienyl, each optionally substituted with methyl or phenyl, and
R⁵ is COOH.

3. The method of claim 1, wherein the compound of formula (I) is a compound of formula (II): 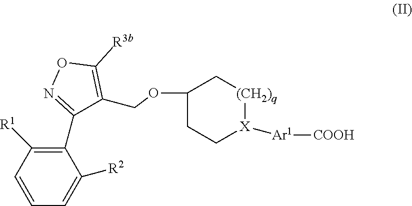
wherein: q is 1 or 2;
R¹ is chloro, fluoro, or trifluoromethoxy;
R² is hydrogen, chloro, fluoro, or trifluoromethoxy;
R^{3b} is trifluoromethyl, cyclopropyl, or isopropyl;
X is CH or N, provided that when X is CH, q is 1;
Ar¹ is selected from benzoisothiazolyl, benzothienyl, indazolyl, indolyl, naphthyl, phenyl and pyridinyl, each optionally substituted with methyl or phenyl.

4. The method according to claim 1, wherein R¹ is chloro or trifluoromethoxy and R² is hydrogen or chloro.

5. The method according to any one of claims 1 to 3, wherein R¹ and R² are both Chloro or wherein R¹ is trifluoromethoxy and R² is hydrogen.

6. The method according to any one of claims 1 to 3, wherein R^{3b} is cyclopropyl or isopropyl.

7. The method according to any one of claims 1 to 3, wherein R^{3b} is cyclopropyl.

8. The method according to any one of claims 1 to 3, wherein Ar¹ is 6-benzoisothiazolyl, 5-benzothienyl, 6-benzothienyl, 6-indazolyl, 5-indolyl or 6-indolyl, A-phenyl and 2-pyridinyl, each optionally substituted with methyl or phenyl.

9. The method according to any one of claims 1 to 3, wherein Ar¹ is 6-benzoisothiazolyl, 5-benzothienyl, 6-benzothienyl, 6-indazolyl, 5-indolyl, 6-indolyl, or 4-phenyl, each optionally substituted with methyl.

10. The method according to any one of claims 1 to 3, wherein Ar¹ group is 5-benzothienyl, 6-benzothienyl, 5-indolyl, 6-indolyl or 4-phenyl, each optionally substituted with methyl.

11. The method according to any one of claims 1 to 3, wherein q is 1 and X is N.

12. The method according to any one of claims 1 to 3, wherein q is 1 and X is CH.

13. The method according to any one of claims 1 to 3, wherein q is 2 and X is N.

14. The method according to any one of claims 1 to 3, wherein R¹ is chloro or trifluoromethoxy; R² is hydrogen or chloro; R³ is cyclopropyl; X is CH or N and Ar¹ group is 4-phenyl, 2-pyridinyl, 6-indolyl or 6-benzothienyl each optionally substituted with methyl.

15. The method according to any one of claims 1 to 3, wherein the compound is selected from:
5-{4-[5-Cyclopropyl-3-(2,6-dichloro-phenyl)-isoxazol-4-ylmethoxy]-piperidin-1-yl}-biphenyl-2-carboxylic acid,
5-{4-[5-Cyclopropyl-3-(2-trifluoromethoxy-phenyl)-isoxazol-4-ylmethoxy]-piperidin-1-yl}-biphenyl-2-carboxylic acid,
5-{4-[3-(2,6-Dichloro-phenyl)-5-isopropyl-isoxazol-4-ylmethoxy]-piperidin-1-yl}-biphenyl-2-carboxylic acid,
4-{4-[3-(2,6-Dichloro-phenyl)-5-isopropyl-isoxazol-4-ylmethoxy]-piperidin-1-yl}-naphthalene-1-carboxylic acid,
4-{4-[5-Cyclopropyl-3-(2,6-dichloro-phenyl)-isoxazol-4-ylmethoxy]-piperidin-1-yl}-3-methyl-benzoic acid,
4-{4-[5-Isopropyl-3-(2-trifluoromethoxy-phenyl)-isoxazol-4-ylmethoxy]-piperidin-1-yl}-benzoic acid,
4-{4-[5-Cyclopropyl-3-(2,6-dichloro-phenyl)-isoxazol-4-ylmethoxy]-piperidin-1-yl}-benzoic acid,
4-{4-[5-Cyclopropyl-3-(2-trifluoromethoxy-phenyl)-isoxazol-4-ylmethoxy]-piperidin-1-yl}-2-methyl-benzoic acid,
4-{4-[5-Cyclopropyl-3-(2,6-dichloro-phenyl)-isoxazol-4-ylmethoxy]-piperidin-1-yl}-2-methyl-benzoic acid,
4-{4-[3-(2,6-Dichloro-phenyl)-5-isopropyl-isoxazol-4-ylmethoxy]-piperidin-1-yl}-2-methyl-benzoic acid,
4-{4-[3-(2,6-Dichloro-phenyl)-5-isopropyl-isoxazol-4-ylmethoxy]-piperidin-1-yl}-benzoic acid,
6-{4-[3-(2,6-Dichloro-phenyl)-5-isopropyl-isoxazol-4-ylmethoxy]-piperidin-1-yl}-1-methyl-1H-indole-3-carboxylic acid,
6-{4-[3-(2,6-Dichloro-phenyl)-5-isopropyl-isoxazol-4-ylmethoxy]-piperidin-1-yl}-benzo[b]thiophene-3-carboxylic acid,
6-{4-[5-Cyclopropyl-3-(2-trifluoromethoxy-phenyl)-isoxazol-4-ylmethoxy]-piperidin-1-yl}-1-methyl-1H-indole-3-carboxylic acid,
6-{4-[5-Cyclopropyl-3-(2,6-dichloro-phenyl)-isoxazol-4-ylmethoxy]-piperidin-1-yl}-1-methyl-1H-indole-3-carboxylic acid,
6-{4-[5-Cyclopropyl-3-(2,6-dichloro-phenyl)-isoxazol-4-ylmethoxy]-piperidin-1-yl}-benzo[b]thiophene-3-carboxylic acid,
4-[5-Cyclopropyl-3-(2,6-dichloro-phenyl)-isoxazol-4-ylmethoxy]-3,4,5,6-tetrahydro-2H-[1,2′]bipyridinyl-5′-carboxylic acid,
4-{4-[3-(2,6-Dichloro-phenyl)-5-isopropyl-isoxazol-4-ylmethoxy]-piperidin-1-yl}-3-methyl-benzoic acid,
4-{4-[5-Cyclopropyl-3-(2,6-dichloro-phenyl)-isoxazol-4-ylmethoxy]-azepan-1-yl}-benzoic acid,
6-{4-[5-Cyclopropyl-3-(2,6-dichloro-phenyl)-isoxazol-4-ylmethoxy]-azepan-1-yl}-1-methyl-1H-indole-3-carboxylic acid,
6-{4-[5-Cyclopropyl-3-(2,6-dichloro-phenyl)-isoxazol-4-ylmethoxy]-azepan-1-yl}-benzo[b]thiophene-3-carboxylic acid,
Trans-4-{4-[5-Cyclopropyl-3-(2,6-dichloro-phenyl)-isoxazol-4-ylmethoxy]-cyclohexyl}-benzoic acid,
Trans-4-{4-[5-Cyclopropyl-3-(2-trifluoromethoxy-phenyl)-isoxazol-4-ylmethoxy]-cyclohexyl}-benzoic acid,
Trans-6-{4-[5-Cyclopropyl-3-(2-trifluoromethoxy-phenyl)-isoxazol-4-ylmethoxy]-cyclohexyl}-1-methyl-1H-indole-3-carboxylic acid, and
Cis-6-{4-[5-Cyclopropyl-3-(2-trifluoromethoxy-phenyl)-isoxazol-4-ylmethoxy]-cyclohexyl)-1-methyl-1H-indole-3-carboxylic acid.

16. The method according to any one of claims 1 to 3, wherein the compound is: 4-(4-[5-Cyclopropyl-3-(2,6-dichloro-phenyl)-isoxazol-4-ylmethoxy]-azepan-1-yl}-benzoic acid: 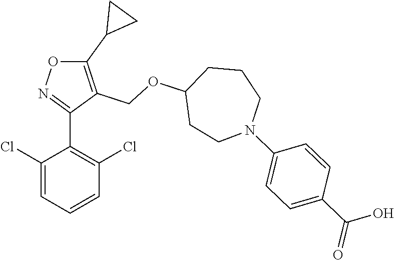 [SMILES: O=C(O)c1ccc(N2CCCC(OCc3c(-c4c(Cl)cccc4Cl)noc3C3CC3)CC2)cc1]
or a pharmaceutically acceptable salt or enantiomer thereof.

17. The method according to any one of claims 1 to 3, wherein the compound is: trans-4-{4-[5-Cyclopropyl-3-(2,6-dichloro-phenyl)-isoxazol-4-ylmethoxy]-cyclohexyl}-benzoic acid: 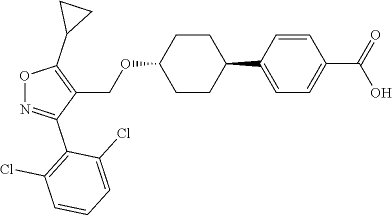 [SMILES: O=C(O)c1ccc([C@H]2CC[C@H](OCc3c(-c4c(Cl)cccc4Cl)noc3C3CC3)CC2)cc1]
or a pharmaceutically acceptable salt or enantiomer thereof.

18. The method according to any one of claims 1 to 3, wherein the compound is: 6-{4-[5-Cyclopropyl-3-(2,6-dichloro-phenyl)-isoxazol-4-ylmethoxy]-piperidin-1-yl}-1-methyl-1H-indole-3-carboxylic acid: 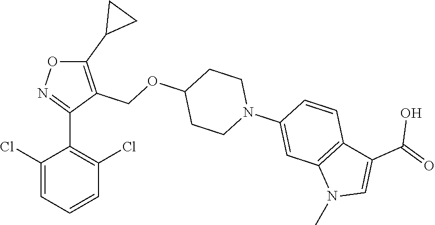 [SMILES: Cn1cc(C(=O)O)c2ccc(N3CCC(OCc4c(-c5c(Cl)cccc5Cl)noc4C4CC4)CC3)cc21]
or a pharmaceutically acceptable salt or enantiomer thereof.

19. The method according to any one of claims 1 to 3, wherein the liver disorder is non-alcoholic steatohepatitis (NASH).

20. The method according to any one of claims 1 to 3, wherein the liver disorder is non-alcoholic fatty liver disease (NAFLD).

21. The method according to any one of claims 1 to 3, wherein the liver disorder is liver inflammation.

22. The method according to any one of claims 1 to 3, wherein the liver disorder is liver fibrosis.

23. The method according to any one of claims 1 to 3, wherein the liver disorder is alcohol induced fibrosis.

24. The method according to any one of claims 1 to 3, wherein the liver disorder is alcoholic steatosis.

25. The method according to any one of claims 1 to 3 of impeding or slowing the progression of non-alcoholic fatty liver disease (NAFLD) to non-alcoholic steatohepatitis (NASH).

26. The method according to any one of claims 1 to 3 of impeding or slowing the progression of NASH.

27. The method according to any one of claims 1 to 3, wherein the therapeutically effective amount is 5 mg/day/patient-600 mg/day/patient.

28. The method according to any one of claims 1 to 3, wherein the compound is administered once daily or twice daily.

29. The method according to any one of claims 1-3, wherein the compound is administered as a pharmaceutically acceptable composition comprising at least one pharmaceutically acceptable excipient, carrier, or diluent.

30. A pharmaceutically acceptable composition comprising a compound of formula (I): 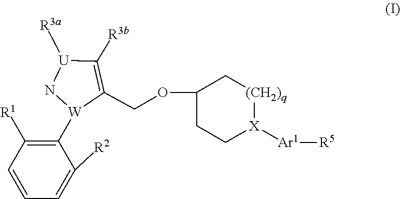
wherein: q is 1 or 2;
U is O, N or C;
W is C or N; provided that when U is O or N, R^{3a} is absent; and provided that when U is N or C, the UN bond is a double bond; and provided that when W is C, the WN bond is a double bond;
X is CH or N;
R¹ is halo or C₁-C₃ alkoxy optionally substituted with 1-5 halo, preferably fluoro atoms;
R² is hydrogen, halo or C₁-C₃ alkoxy optionally substituted with 1-5 halo, preferably fluoro atoms;
R^{3a} is hydrogen, or is absent;
R^{3b} is C₁-C₃ alkyl optionally substituted with 1-5 halo, preferably fluoro atoms; or is C₃-C₄ cycloalkyl optionally substituted with 1-3 methyl or ethyl groups; or is a 4 membered heterocyclyl optionally substituted with 1-3 methyl or ethyl groups;
Ar¹ is selected from optionally substituted 6-10 member aryl, optionally substituted 5-10 membered heteroaryl; and
R⁵ is COOH or a carboxylic acid isostere;
or a tautomer thereof, or an isotopomer of each thereof, or an enantiomer or diastereomer of the foregoing, or a pharmaceutically acceptable salt of each of the above; and
at least one pharmaceutically acceptable excipient, carrier, or diluent for treating a liver disorder; impeding or slowing the progression of non-alcoholic fatty liver disease (NAFLD) to non-alcoholic steatohepatitis (NASH); or for impeding or slowing the progression of NASH.

31. The pharmaceutically acceptable composition of claim 30, wherein:
q is 1 or 2, provided that when X is CH, q is 1;
U is O, N or C; provided that when U is O or N, R^{3a} is absent; and provided that when U is N or C, the UN bond is a double bond; and provided that when W is C, the WN bond is a double bond;
W is C or N;
X is CH or N;
R¹ is chloro, fluoro, or trifluoromethoxy;
R² is hydrogen chloro, fluoro, or trifluoromethoxy;
R^{3a} is hydrogen, or absent;
R^{3b} is trifluoromethyl, cyclopropyl or isopropyl;
Ar¹ is selected from optionally substituted indolyl, optionally substituted benzothienyl, optionally substituted naphthyl, optionally substituted phenyl, optionally substituted benzoisothiazolyl, optionally substituted indazolyl, and optionally substituted pyridinyl; preferably, indolyl, benzothienyl, naphthyl, phenyl, benzoisothiazolyl, indazolyl, and pyridinyl, each optionally substituted with a group selected from methyl, ethyl, and phenyl; more preferably 6-indolyl, 6-benzothienyl, 4-naphthyl, 4-phenyl, and 2-pyridinyl, each optionally substituted with one or two groups independently selected from methyl, ethyl, and phenyl; yet more preferably 4-phenyl, 6-indolyl or 6-benzothienyl, each optionally substituted with methyl or phenyl, and
R⁵ is COOH.

32. The pharmaceutically acceptable composition of claim 30, wherein the compound of formula (I) is a compound of formula (II): 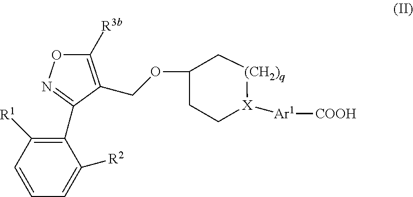
wherein: q is 1 or 2;
R¹ is chloro, fluoro, or trifluoromethoxy;
R² is hydrogen, chloro, fluoro, or trifluoromethoxy;
R^{3b} is trifluoromethyl, cyclopropyl, or isopropyl;
X is CH or N, provided that when X is CH, q is 1;
Ar¹ is selected from benzoisothiazolyl, benzothienyl, indazolyl, indolyl, naphthyl, phenyl and pyridinyl, each optionally substituted with methyl or phenyl.

33. A unit dose form of the pharmaceutically acceptable formulation of any one of claims 30-32.
